(19) United States
(12) Patent Application Publication
(10) Pub. No.: US 2024/0341741 A1
(43) Pub. Date: Oct. 17, 2024

(54) TISSUE RETRACTOR HAVING A BEVEL GEAR DRIVEN RETRACTOR BLADE

(71) Applicant: Astura Medical Inc., Iriving, TX (US)

(72) Inventors: Benjamin McPherson, Irving, TX (US); Thomas Purcell, Irving, TX (US)

(73) Assignee: Astura Medical Inc., Irving, TX (US)

(21) Appl. No.: 18/637,400

(22) Filed: Apr. 16, 2024

Related U.S. Application Data
(60) Provisional application No. 63/456,458, filed on Mar. 31, 2023.

(51) Int. Cl. A61B 17/02

(52) U.S. Cl. CPC A61B 17/0218

(57) ABSTRACT

A tissue retractor having a bevel gear driven retractor blade that features a tool-less coarse adjustment that allows a single surgeon to easily manipulate and secure the orientation of a retractor blade while preparing the surgical corridor.

### CROSS-REFERENCE TO RELATED APPLICATIONS

This application claims the benefit of U.S. Provisional Application No. 63/456,458 filed Mar. 31, 2023, which is incorporated herein by reference.

### FIELD

The present invention relates generally to the field of surgery, and more specifically, to a tissue retractor for use in Spinal Fusion Surgery.

### BACKGROUND

Many types of spinal surgeries require exposure and access through the skin to internal parts of the body (“surgical area”). Tissue retractors are typically used to create openings at the surgical area of sufficient size to allow the surgeon ample access for carrying out procedures Tissue retractors are typically mechanized devices designed to generate a pathway through tissue using retractor blades for surgical access and to remain open during the surgery and allow the surgeon to perform the desired procedure.

One procedure being used is an anterior lumbar approach to the spine for anterior lumbar interbody fusion (ALIF). Current competitive access systems for the anterior lumbar approach require a coordinated effort between the surgeon and assistants to place and orient the retractor blades within the abdominal cavity. Delicate structures, such as veins and organ tissue, must be carefully mobilized under constant visualization which requires minimal interaction with the access system retractor blades to avoid distraction which could lead to surgical complications.

Some problems with the current access systems include Retractor Blade Angle Adjustment and Fixation, and Blade drift.

Retractor Blade Angle Adjustment and Fixation: To navigate through an abdominal surgical corridor and mobilize delicate tissue, a full 6-degrees of freedom at the retractor blade is necessary in order to place the retractor blade in the optimal position, which is dictated by the structure of the retracted tissue. Once in position, the retractor blade must be locked in place. Current access systems require a two-step locking procedure to secure the retractor blades; the retractor blade angle relative to the supporting shaft must be locked at the knuckle via a screw that requires a driver followed by the fixation of the arm at the support frame/carriage to lock down the remaining degrees of freedom.

Retractor Blade Drift: Current access systems use a friction based locking system, such as a screw pressing into a face or a screw-clamping collar around a shaft, to hold the retractor blade angle relative to the supporting shaft. A friction based locking system can slip in situations where the retracted tissue places a significant amount of force on the retractor blade, causing the retractor blade angle to drift from the desired position.

Some competitors offer surgeons numerous devices that retract abdominal tissue to gain access to the spine for spinal fusion surgery. Some retractor system offerings do not allow a tool-less retractor blade set-up and adjustment during tissue retraction. A driver or tool is required to modify and/or secure the tilt angle of the retractor blade relative to the supporting arm.

To change and/or secure the retractor blade orientation during the access procedure, either a second pair of hands is required, or the surgeon's attention must be diverted from the immediate surgical site, increasing the risk of tissue damage. A convenient method does not exist by which the angle of the retractor blade can be easily adjusted and secured in orientation with the use of just two hands.

Accordingly, there remains a need for instruments and methods that provide a faster, more convenient method of adjusting and locking the retractor blade position is needed to minimize distraction during this critical phase of the access procedure. The present invention is directed toward meeting these needs.

### SUMMARY

The present invention is directed to a tissue retractor having a bevel gear driven retractor blade that features a tool-less coarse adjustment that allows a single surgeon to easily manipulate and secure the orientation of a retractor blade while preparing the surgical corridor. During the initial setting of the retractor blades, a large degree of tilting movement is needed to orient the blade relative to the tissues being retracted. The surgeon can easily change the blade angle, and have it maintain that angle, by rotating the adjustment handle with their fingers. Current offerings require the blade be in a floating or unlocked state while setting the initial blade position followed by a tool or driver to lock the orientation. Once in position, the retractor blade angle is adjusted while under load for further retraction. The bevel gear driven retractor blade features a leveraged fine adjustment via the pinion gear driver profile to facilitate this. A driver can be used to rotate the pinion gear set, which turns the pinion gear to change the angle of the retractor blade. This pinion gear set is located adjacent to the retractor blade to allow the surgeon to maintain visual of the surgical site.

### BRIEF DESCRIPTION OF THE DRAWINGS

FIG. 1 is a perspective view of a tissue retractor having a bevel gear driven retractor blade.

FIG. 2 is a perspective view of the distal portion of the tissue retractor of FIG. 1.

FIGS. 3A and 3B are sectional views showing more detail of the retractor blade adjustment mechanism and blade carrier.

FIG. 4 shows the tissue retractor connected to a retractor support assembly.

### DETAILED DESCRIPTION

The present invention is directed to systems, methods, and devices applicable to spinal surgery. More specifically, the present invention is directed to a tissue retractor that is designed to change a retractor blade angle while preparing the surgical corridor and when the retractor blade is in position. During the initial setting of the retractor blade, a large degree of tilting movement is needed to orient the blade relative to the tissues being retracted. This adjustment is accomplished using an adjustment handle that allows a single surgeon to easily manipulate and secure the orientation of a retractor blade while preparing the surgical corridor. The surgeon can easily change the retractor blade angle, and have it maintain that angle, by rotating the adjustment handle with their fingers. Once in position, the retractor blade angle may be further adjusted while under load for further retraction. The tissue retractor features a leveraged fine-tune adjustment via the pinion gear driver profile to facilitate this. A driver can be used to rotate the pinion gear set to change the angle of the blade relative to the support shaft. This pinion gear set adjustment feature is located adjacent to the retractor blade to allow the surgeon to maintain visual of the surgical site while making the retractor blade angle adjustment.

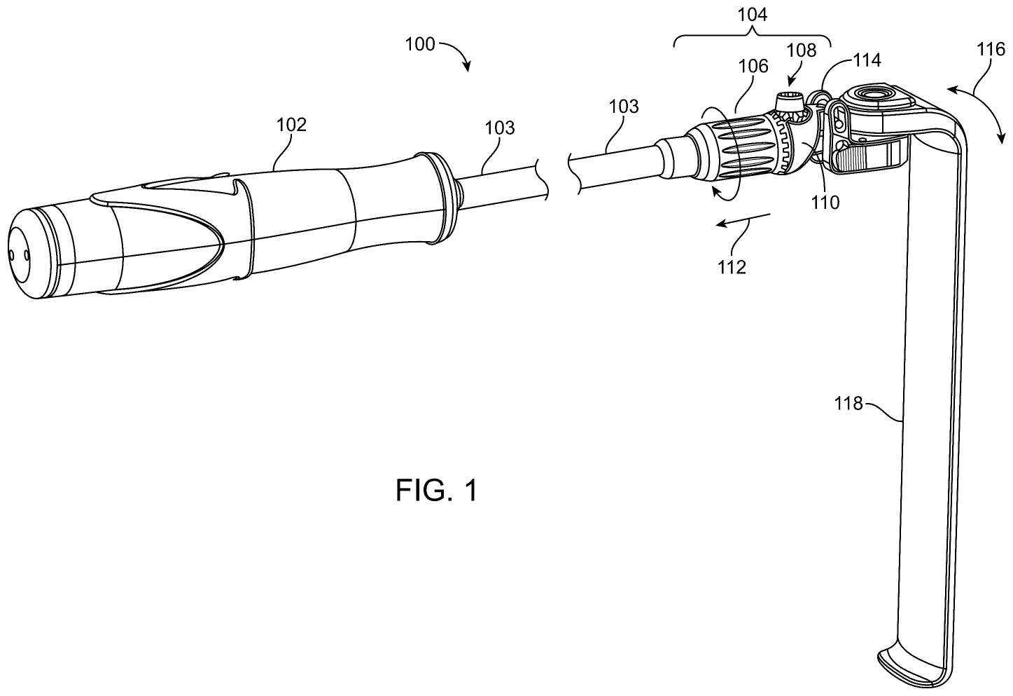

FIG. 1 shows a perspective view of a first embodiment of a tissue retractor 100 having a handle 102 coupled to a retractor blade adjustment mechanism 104, the retractor blade adjustment mechanism 104 having a first adjustment mechanism 106 and a second adjustment mechanism 108. The retractor blade adjustment mechanism 104 is coupled to a retractor blade carrier 114, and the retractor blade carrier 114 is attached to a retractor blade 118. In use, the first adjustment mechanism 106 is configured to rotate or pivot the retractor blade 118 a large degree 116 for gross adjustment, and the second adjustment mechanism 108 is configured to rotate or pivot the retractor blade 118 a small degree 116 for fine adjustment.

The surgeon can easily change the retractor blade angle 116 a large degree with the retractor blade adjustment mechanism 104, and have it maintain that angle, by rotating the first adjustment mechanism 106, and once in position, rotating the second adjustment mechanism 108 for further retraction while under load a small degree to fine-tune the angle of the retractor blade 118.

In other embodiments, the handle 102 is coupled to a proximal end of a shaft 103, and a distal end of the shaft 103 is coupled to the retractor blade adjustment mechanism 104. The first adjustment mechanism 106 includes an adjustment handle 106, and the second adjustment mechanism 108 includes a pinion gear set 108. The adjustment handle 106 and pinion gear set 108 are coupled to a translating shuttle 110 that is designed to rotate or pivot a retractor blade carrier 114 to change an angle 116 of a retractor blade 118.

In use, the adjustment handle 106 is used to quickly achieve a large degree of tilting movement needed to orient the retractor blade 118 relative to the tissues being retracted. The adjustment handle 106 allows a single surgeon to easily manipulate and secure the orientation of a retractor blade 118 while preparing the surgical corridor. The surgeon can easily change the retractor blade angle 116, and have it maintain that angle, by rotating the adjustment handle 106 with mating internal gear with their fingers. Once in position, the retractor blade angle 116 relative to the supporting shaft may be further adjusted, fine-tuned, while under load for further retraction. The tissue retractor 100 features a leveraged fine-tune adjustment via the pinion gear driver profile to facilitate this. A driver can be used to rotate the pinion gear set 108 to change the angle of the blade 116 relative to the square shaft 124. This pinion gear set 108 adjustment feature is located adjacent to the retractor blade 118 to allow the surgeon to maintain visual of the surgical site while making the retractor blade angle adjustment 116.

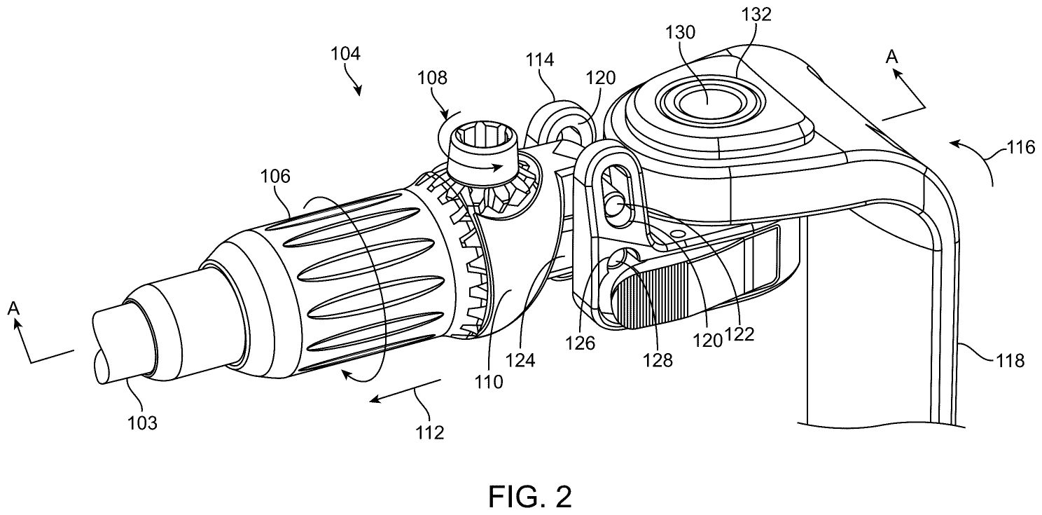

FIG. 2 is a perspective view of the distal portion of the tissue retractor 100 showing the retractor blade adjustment mechanism 104 having the adjustment handle 106 coupled to the pinion gear set 108 and translating shuttle 110. The distal end of the translating shuttle 110 is coupled to slotted protrusions 120 of the blade carrier 114 with an upper pin 122. The adjustment handle 106 includes a threaded inner diameter coupled to a threaded portion of a square shaft 124. The distal end of the square shaft 124 is rotatably coupled to the blade carrier 114 with a lower pin 128. The lower pin 128 is the hinge about which the blade carrier 114 pivots. The upper pin 122 applies the forward or backward force to the slotted protrusions 120 via the translating shuttle 114 to change the angle 116 of the blade carrier 114 relative to the square shaft 124. The blade carrier 114 is coupled with the blade 118 by known means, such as a pin or post 130 on the blade carrier 114 engaging an engagement hole 132 of the retractor blade 118.

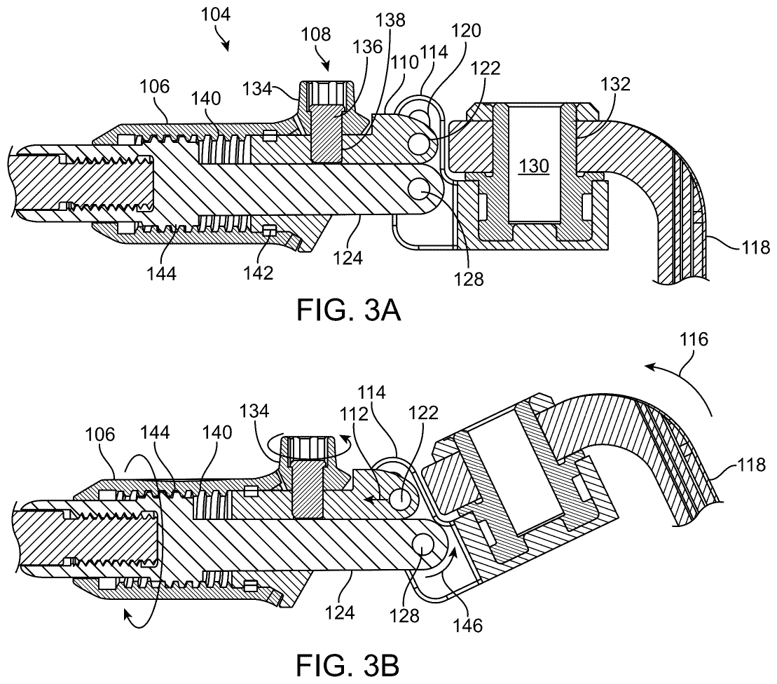

FIGS. 3A and 3B are sectional views at A-A of FIG. 2 showing more detail of the retractor blade adjustment mechanism 104 and blade carrier 114. FIG. 3A shows the retractor blade in a first position and FIG. 3B shows the retractor blade in a rotated second position.

The bevel gear driven blade adjustment mechanism 114 includes a pinion gear set 108 affixed to a translating shuttle 110 that changes the angle 116 of a retractor blade 118. The pinion gear set 108 includes a pinion gear 134, featuring a driver profile, is secured to the translating shuttle 110 by a headed pin 136 through a bore of the pinion 134 into a hole 138 of the translating shuttle 110. The adjustment handle 104 includes a threaded internal diameter 140 and is secured to the translating shuttle 110 by a captured c-ring 142 that allows adjustment handle 104 to rotate about the translating shuttle 110.

The physical dimensions of a bevel gear-set are determined by the number of teeth on each gear, pressure angle, tooth module, and shaft angle. These inputs must be tailored in a specific manner to meet the desired size of the mechanism while maintaining functional requirements such as the gear ratio and tooth load capacity. The bevel gear-set is incorporated directly into the design of the driving and driven components of the mechanism for a compact envelope. The use of standard gear-sets would increase the complexity and size of the mechanism along with a less desirable form factor.

The translating shuttle 110 and pinion gear set 108 translate together along the square shaft 124 having a threaded section 144 engaging the threaded internal diameter 140 of the adjustment handle. The mating thread of the threaded internal diameter 140 and the square shaft 124 allows linear translation 112 of the translating shuttle 110 along the square shaft 124 while also maintaining position of the translating shuttle 110 if the pinion gear 134 is not rotated.

The translating shuttle 110 is pinned to the blade carrier 114 via the slotted protrusion 120 at the blade-end of the translating shuttle 110. The blade carrier 114 is secured to the square shaft 124 and translating shuttle 110 with upper and lower pressed pins. The lower pin 128 is the hinge about which the blade carrier 114 pivots 146. The upper pin 122 applies the forward or backward force via the slotted protrusions 120 of the blade carrier 114 to change the angle 116 of the blade carrier 114 relative to the square shaft 124.

In use, the square shaft 124 remains stationary and the translating shuttle 110 along with the attached pinion gear set 108 moves along the square shaft 124 as an assembly which pushes and pulls on the top pin 122 at the blade carrier 114 to adjust the retractor blade angle 116. Turning the translating shuttle 110 larger gear (with one's fingers) allows for larger blade movement per revolution and turning the smaller pinion gear 134 with a driver coupled with the headed pin 136 allows for finer retractor blade movement with additional leverage per revolution.

The gear driven adjustment system allows for quick, course, adjustment of the retractor blade angle by hand while positioning the blades in the surgical corridor. Additional leverage and fine adjustment to the blade angle can be made via the pinion gear with a driver.

Using a course and fine adjustment of the retractor blade angle in a single mechanism allows the surgeon to optimally place the retractors blade in a quick manner while maintaining visual of the surgical area.

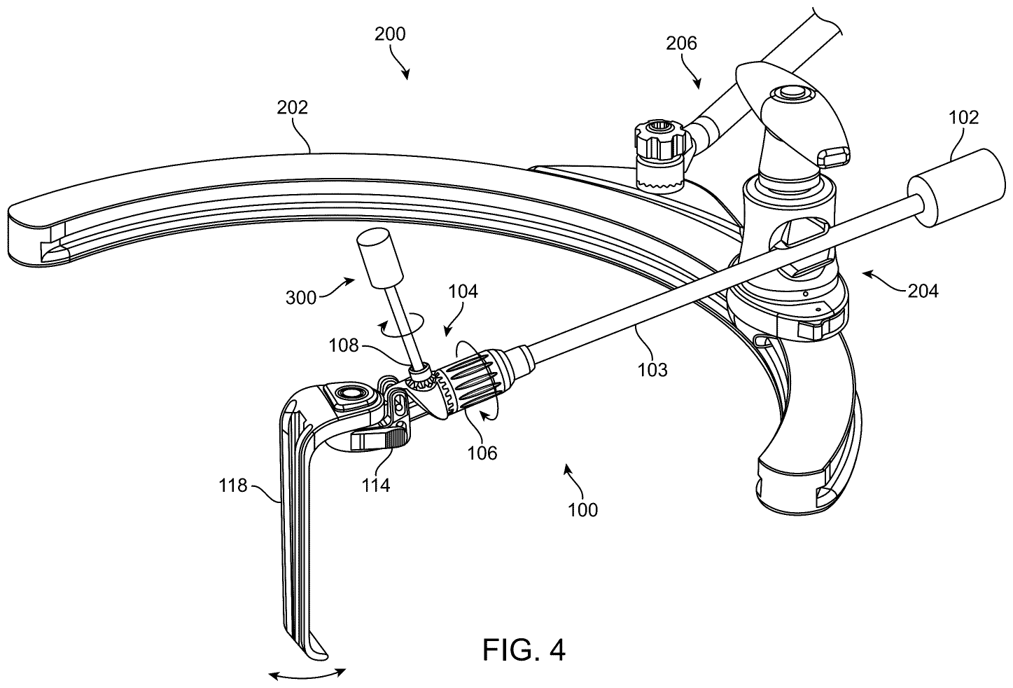

FIG. 4 shows a tissue retractor assembly 200 that is designed hold the tissue retractor 100 over a patent to create openings at the surgical area sufficient size to allow the surgeon ample access for carrying out procedures. The tissue retractor assembly 200 includes a frame 202 with a tissue retractor clamping mechanism 204 and table arm assembly 206 to secure the tissue retractor assembly 200 and frame 204 in the correct position above the surgical area. The retractor claim is coupled with the clamping mechanism 202 that designed to hold the tissue retractor 100 in the correct position for the retractor blade 118 reach the opening in the surgical area and retract the tissue the desired distance by moving the retractor blade 118.

Retraction of the tissue is done using the adjustment handle 106 and pinion gear set 108. The adjustment handle 106 allows a single surgeon to easily manipulate and secure the orientation of a retractor blade 118 while preparing the surgical corridor. The surgeon changes the retractor blade angle 116, and have it maintain that angle, by rotating the adjustment handle 106 with their fingers. Once in position, the retractor blade angle 116 is fine-tuned via the pinion gear set 108. A driver 300 can be used to rotate the pinion gear set 108 to change the angle of the blade 118 relative to the support shaft. This pinion gear set adjustment feature 104 is located adjacent to the retractor blade 118 to allow the surgeon to maintain visual of the surgical site while making the retractor blade angle adjustment.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A tissue retractor comprising:
a handle;
a retractor blade adjustment mechanism coupled to the handle having:
a first adjustment mechanism; and
a second adjustment mechanism;
a retractor blade carrier coupled to the retractor blade adjustment mechanism configured to pivot in response to the first and second adjustment mechanisms; and
a retractor blade coupled to the retractor blade carrier;
wherein the first adjustment mechanism is configured to adjust the retractor blade a large degree and the second adjustment mechanism is configured to adjust the retractor blade a small degree.

2. The tissue retractor of claim 1, wherein the first adjustment mechanism includes an adjustment handle and the second adjustment mechanism includes a pinion gear set.

3. The tissue retractor of claim 2, wherein the retractor blade adjustment mechanism further includes a translating shuttle using the adjustment handle and/or pinion gear set to change the angle of the retractor blade.

4. A tissue retractor comprising:
a retractor blade coupled to a retractor blade carrier;
a retractor blade adjustment mechanism coupled to the retractor blade carrier configured to change a retractor blade angle, the retractor blade adjustment mechanism includes:
a first adjustment mechanism configured to pivot the retractor blade a first angle; and
a second adjustment mechanism configured to pivot the retractor blade a second angle.

5. The tissue retractor of claim 1, wherein the first adjustment mechanism is configured to pivot the retractor blade a large degree for gross adjustment, and the second adjustment mechanism is configured to pivot the retractor blade a small degree for fine adjustment.

6. The tissue retractor of claim 1, wherein the first adjustment mechanism includes an adjustment handle, and the second adjustment mechanism includes a pinion gear set.

7. The tissue retractor of claim 6, wherein the adjustment handle and pinion gear set are coupled to a translating shuttle that is designed to pivot the retractor blade carrier to change the angle the of a retractor blade.

8. The tissue retractor of claim 6, further comprising a driver configured to rotate the pinion gear set to change the angle of the retractor blade.

9. A tissue retractor comprising:
a retractor blade coupled to a retractor blade carrier;
a retractor blade adjustment mechanism coupled to the retractor blade carrier configured to change a retractor blade angle, the retractor blade adjustment mechanism includes:
a first adjustment mechanism having an adjustment handle configured to pivot the retractor blade a large degree for gross adjustment; and
a second adjustment mechanism having a pinion gear set configured to pivot the retractor blade a small degree for fine adjustment.

10. The tissue retractor of claim 9, wherein the adjustment handle and pinion gear set are coupled to a translating shuttle that is designed to pivot the retractor blade carrier to change the angle the of a retractor blade.

11. The tissue retractor of claim 9, further comprising a driver configured to rotate the pinion gear set to change the angle of the retractor blade.
